(19) United States
(12) Patent Application Publication
(10) Pub. No.: US 2020/0258107 A1
(43) Pub. Date: Aug. 13, 2020

(54) REWARDS PROGRAM METHOD AND DEVICES

(71) Applicant: Richard Bruno Muller, Marietta, GA (US)

(72) Inventor: Richard Bruno Muller, Marietta, GA (US)

(21) Appl. No.: 16/271,801

(22) Filed: Feb. 9, 2019

(51) Int. Cl. G06Q 30/02; G16H 50/30

(52) U.S. Cl. CPC G06Q 30/0215; G16H 50/30

(57) ABSTRACT

The embodiments disclose a method including establishing a worldwide rewards program with a digital network for receiving credit card holder member data and providing interactive services and functions to members, contracting with companies on a participation basis for funding the credit card holder member rewards, awarding rewards purchase points to a credit card holder member for use in redeeming rewards, offering purchases of contracted companies products including vehicles using credit card holder member points for redemption purchases, and personalizing member data authorized by members using a rewards digital device application.

### BACKGROUND

Many major credit card issuers are offering higher reward points for goods and services as well as Cash Back deals. Many of these new offers contain limits, timeframe requirements and restrictions. A cardholder uses or loses reward opportunity not necessarily according to when or on what they need or want but just to not lose a reward opportunity. Many of these reward offerings are limited to specific geographical locales and should a cardholder use the credit card in another country no rewards points are awarded.

### BRIEF DESCRIPTION OF THE DRAWINGS

FIG. 1 shows a block diagram of an overview flow chart of rewards program method and devices of one embodiment.

FIG. 2A shows a block diagram of an overview of rewards program credit card cardholder members of one embodiment.

FIG. 2B shows a block diagram of an overview of contracting with companies in a rewards program of one embodiment.

FIG. 3 shows a block diagram of an overview of rewards program digital network of one embodiment.

FIG. 4 shows a block diagram of an overview of rewards program digital device application of one embodiment.

FIG. 5 shows for illustrative purposes only an example of rewards program server of one embodiment.

FIG. 6 shows a block diagram of an overview of rewards program member memberships of one embodiment.

FIG. 7 shows for illustrative purposes only an example of self-reported data OF activities of daily living of one embodiment.

FIG. 8 shows for illustrative purposes only an example of health risk assessment of one embodiment.

FIG. 9 shows for illustrative purposes only an example of tracking your social media of one embodiment.

FIG. 10 shows for illustrative purposes only an example of concierge travel plans of one embodiment.

FIG. 11 shows for illustrative purposes only an example of tracking your visits of one embodiment.

FIG. 12 shows a block diagram of an overview of AutoBuy Earnfast Rewards Program benefits of one embodiment.

FIG. 13 shows a block diagram of an overview of vehicle purchase and related benefits of one embodiment.

FIG. 14A shows a block diagram of an overview of community banking system participant benefits of one embodiment.

FIG. 14B shows a block diagram of an overview of random drawing for members of one embodiment.

FIG. 15A shows a block diagram of an overview of Opt.In Ecosystem program of one embodiment.

FIG. 15B shows a block diagram of an overview of AutoBuy Earnfast Rewards Program worldwide of one embodiment.

FIG. 16A shows a block diagram of an overview of AutoBuy Rewards Program revenue streams of one embodiment.

FIG. 16B shows a block diagram of an overview of AutoBuy Rewards program micro-payments continued of one embodiment.

FIG. 16C shows a block diagram of an overview of escrow account earned interest commissions of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the embodiments.

### General Overview:

It should be noted that the descriptions that follow, for example, in terms of a rewards program method and devices is described for illustrative purposes and the underlying system can apply to any number and multiple types of credit card transactions and product and service benefits. In one embodiment of the present invention, the rewards program method and devices can be configured using member accumulated points to determine benefits available to a member. The rewards program method and devices can be configured to include OEM manufacturer participants and can be configured to include credit card company participants using the embodiments.

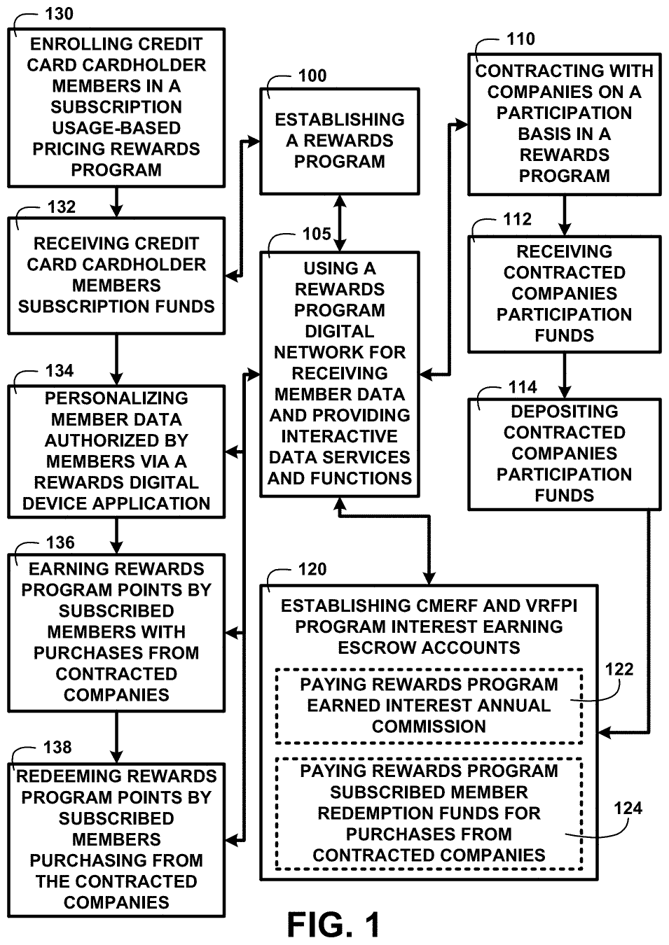

FIG. 1 shows a block diagram of an overview flow chart of rewards program method and devices of one embodiment. FIG. 1 shows establishing a rewards program 100 using a rewards program digital network for receiving member data and providing interactive data services and functions 105. Establishing a rewards program 100 includes contracting with companies on a participation basis in a rewards program 110. Contracted participating companies will execute a first 5 years Agreement/Contract; a second 5 years Agreement/Contract; a third 5 years Agreement/Contract; and one (1) 35 years Contract/Agreement of one embodiment.

Funding the rewards program includes receiving contracted companies participation funds 112. Depositing contracted companies participation funds 114 allows establishing CMERF and VRFPI program interest earning escrow accounts 120. Earning interest with the Car Manufacturers Escrow Redemption Fund (CMERF) and Vendor Reward Fund Participation Income (VRFPI) escrow accounts provides income to the rewards program company for operating by paying rewards program earned interest annual commission 122 to the rewards program company. Paying rewards program subscribed member redemption funds for purchases from contracted companies 124 using the balance of the CMERF and VRFPI program escrow accounts interest earnings can increase member purchases from contracted participating companies. The rewards program includes enrolling credit card cardholder members in a subscription usage-based pricing rewards program 130. Enrolling credit card cardholder members in a subscription usage-based pricing rewards program 130 further is increasing income through receiving credit card cardholder member subscription funds 132. The rewards program can personalize member rewards benefits by personalizing member data authorized by members via a rewards digital device application 134. Learning member likes and dislikes and daily living activities can include for example a members' health condition when providing personalized data used to personalize a members' benefits. Benefits come from earning rewards program points by subscribed members with purchases from contracted companies 136. Redeeming rewards program points by subscribed members purchasing from the contracted companies 138 can reduce subscribed members out of pocket purchasing expenses of one embodiment.

### DETAILED DESCRIPTION

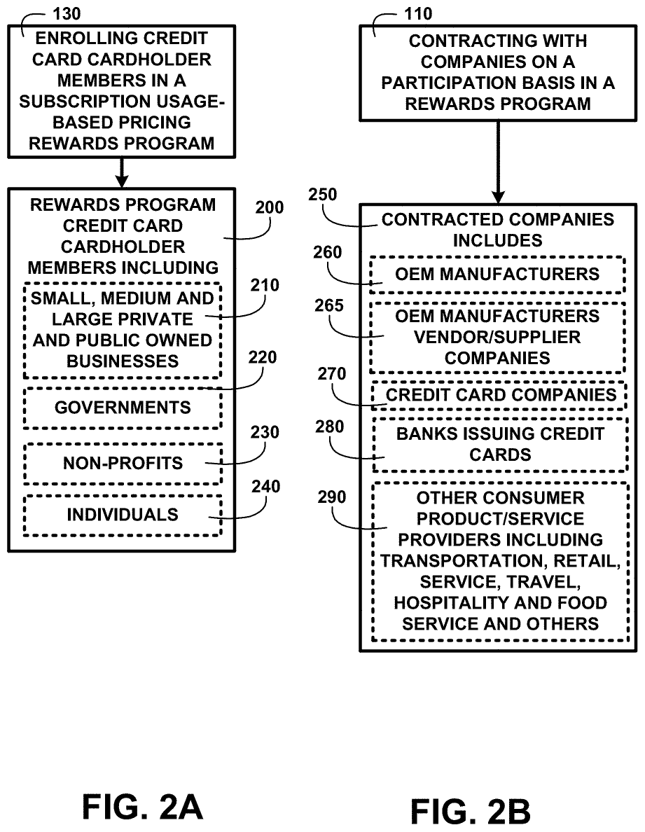

FIG. 2A shows a block diagram of an overview of rewards program credit card cardholder members of one embodiment. FIG. 2A shows enrolling credit card cardholder members in a subscription usage-based pricing rewards program 130. Enrollment of rewards program credit card cardholder members including 200 small, medium and large private and public owned businesses 210, governments 220, non-profits 230 and individuals 240 can lead to a rapid growth of member enrollment of one embodiment.

Contracting with Companies in a Rewards Program:

FIG. 2B shows a block diagram of an overview of contracting with companies in a rewards program of one embodiment. FIG. 2B shows contracting with companies on a participation basis in a rewards program 110 increases the range of contracted company participation funds and member benefits offerings. The rewards program participation contracted companies includes 250 OEM manufacturers 260, OEM manufacturers vendor/supplier companies 265, credit card companies 270, banks issuing credit cards 280, and other consumer product/service providers including transportation, retail, service, travel, hospitality and food service and others 290 of one embodiment.

### Rewards Program Digital Network:

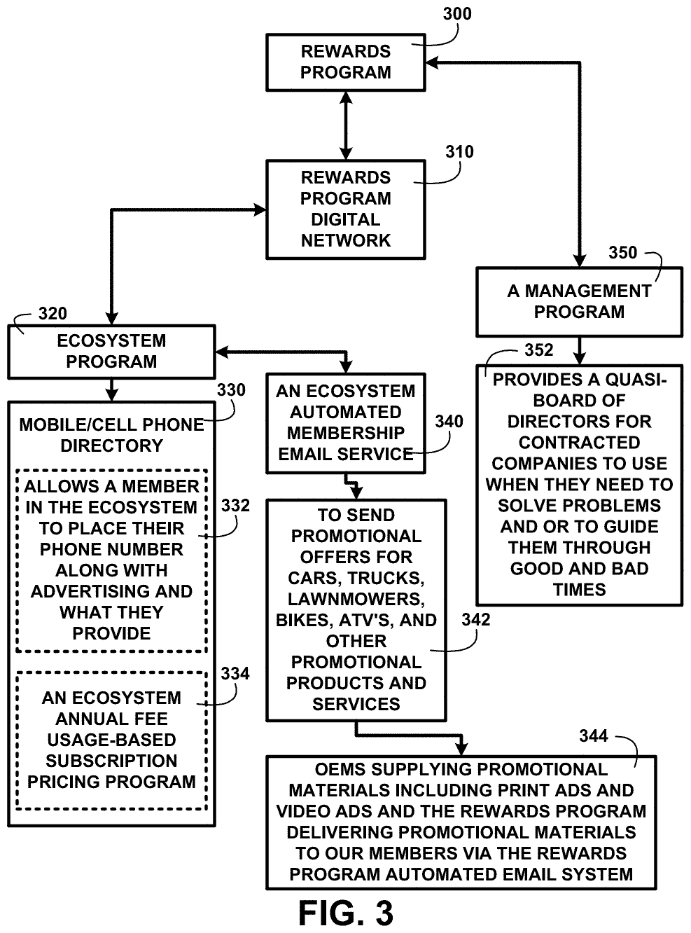

FIG. 3 shows a block diagram of an overview of rewards program digital network of one embodiment. FIG. 3 shows a rewards program 300 including a rewards program digital network 310. The rewards program digital network 310 includes an Ecosystem program 320 including a mobile/cell phone directory 330. The mobile/cell phone directory 330 allows a member in the Ecosystem to place their phone number along with advertising and what they provide 332. The Ecosystem program 320 includes an Ecosystem annual fee usage-based subscription pricing program 334. The rewards program digital network 310 includes an Ecosystem automated membership email service 340 to send promotional offers for cars, trucks, lawnmowers, bikes, ATV's, and other promotional products and services 342 to subscribed members. The rewards program digital network 310 includes OEMs supplying promotional materials including print ads and video ads and the Ecosystem delivering promotional materials to our members via the automated membership email service 344. The rewards program digital network 310 includes a management program 350 element which provides a quasi-board of directors for contracted companies to use when they need to solve problems and or to guide them through good and bad times 352 of one embodiment.

### Rewards Program Digital Device Application:

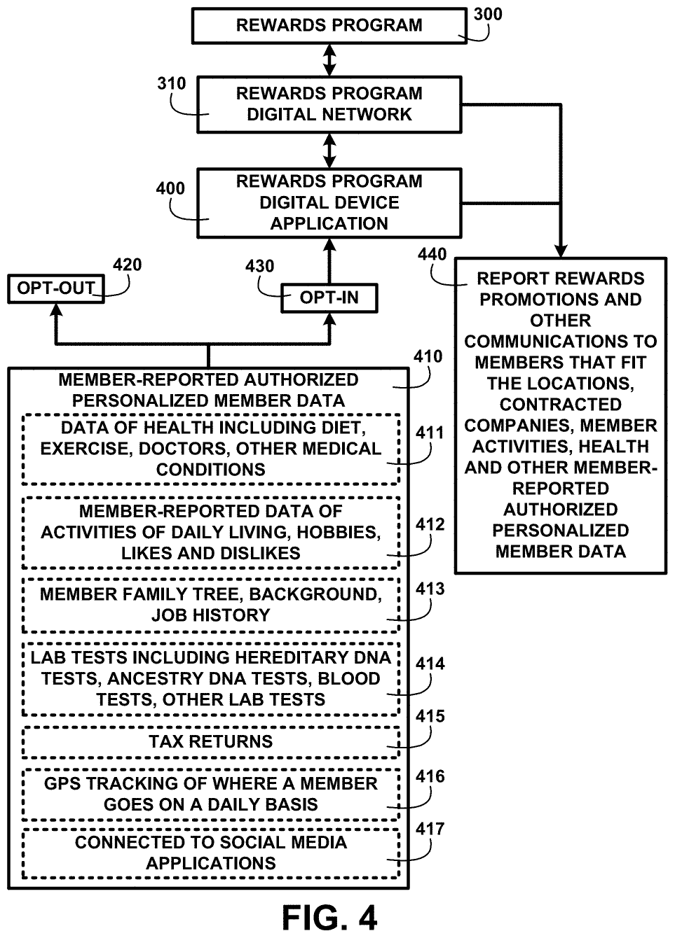

FIG. 4 shows a block diagram of an overview of rewards program digital device application of one embodiment. FIG. 4 shows the rewards program 300 rewards program digital network 310 including a rewards program digital device application 400. The rewards program digital device application 400 can be used to report rewards promotions and other communications to members that fit the locations, contracted companies, member activities, health and other member-reported authorized personalized member data 440. A subscribed member may choose an opt-out 420 selection to not authorize member-reported personalized member data using the rewards program digital device application 400. The subscribed member may choose an Opt-In 430 selection to authorize the gathering of member-reported authorized personalized member data 410 using the rewards program digital device application 400. Authorized personalized member data can include data of health including diet, exercise, doctors, other medical conditions 411, member-reported data of activities of daily living, hobbies, likes and dislikes 412, member family tree, background, job history 413, lab tests including hereditary DNA tests, ancestry DNA tests, blood tests, other lab tests 414, tax returns 415, and GPS tracking of where a member goes on a daily basis 416. The member authorization can include the rewards program digital device application 400 to be connected to social media applications 417 of the subscribed member of one embodiment.

### Rewards Program Server:

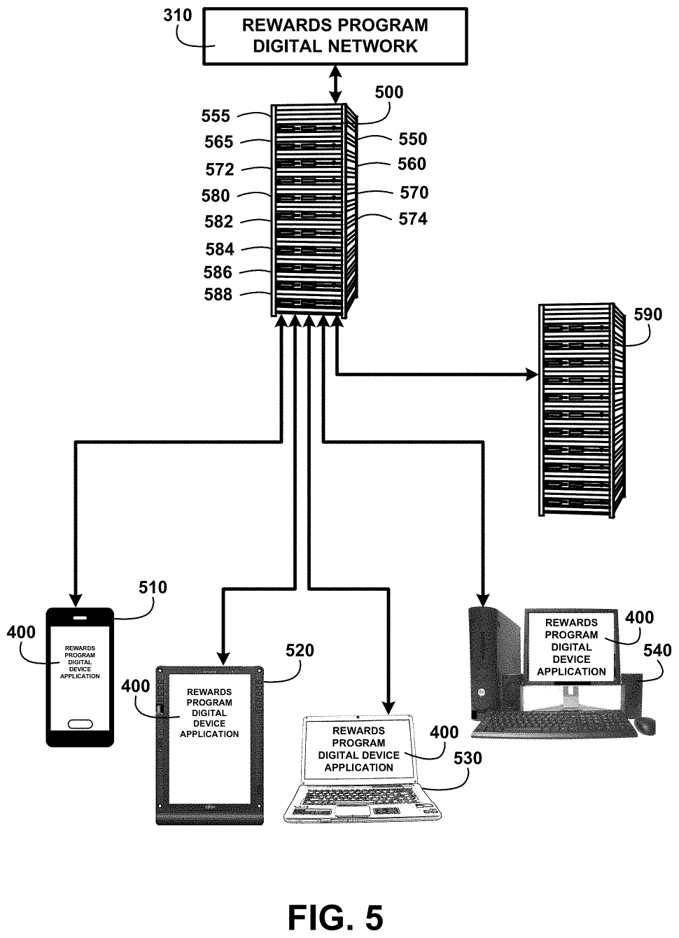

FIG. 5 shows for illustrative purposes only an example of rewards program server of one embodiment. FIG. 5 shows the rewards program digital network 310 including at least one rewards program server 500; at least one digital database 550; at least one digital processor 560; at least one OCR device 570; at least one WI-FI connectable device 574; at least one printer 555; at least one video device 565; at least one language translation apparatus 572; at least one data mining device 580; at least one internet connectable device 582; and voice recognition and transcription device 584; at least one email server 588 and an automated membership email service database 586 of one embodiment.

The at least one digital database 550 can be used for example to record and retrieve authorized personalized member data. The at least one digital processor 560 can be used for example for processing authorized personalized member data to analyze the personalized member data for example to determine a subscribed members' health risk assessment. The at least one OCR device 570 can be used for example for converting a subscribed members' ancestry DNA tests to a readable text for use in determining the subscribed members' health risk assessment. The at least one WI-FI connectable device 574 can be used for example for connecting a user digital device to the at least one rewards program server 500 for gathering authorized personalized member data with the rewards program digital device application 400 of one embodiment.

The at least one printer 555 can be used for example for printing hard copy reports for a contracted company of VRFPI program escrow accounts balances and transactions. The at least one video device 565 can be used for example for recording promotional materials for distribution to a subscribed members using the mobile/cell phone directory 330 and rewards program automated email system 344. The at least one language translation apparatus 572 can be used for example for translating contracted companies promotional materials into a subscribed members' selected default language of one embodiment.

The at least one data mining device 580 can be used for example for gathering up to date information and data on hundreds of brands and models for subscribed member buyers to choose for redemption of reward points purchases. The at least one internet connectable device 582 can be used for example for connecting a user digital device with the rewards program digital device application 400 installed for browsing reward points redemption opportunities of one embodiment.

The rewards program digital network 310 including at least one rewards program server 500 can include an interactive connection to at least one contracted company server 590. The interactive connection to at least one contracted company server 590 can be used for reporting subscribed member purchases; redemption points and participation funds transactions; CMERF and VRFPI program escrow accounts balances and transactions; transfers of promotional materials and other communications and data transfers of one embodiment.

The rewards program digital network 310 at least one rewards program server 500 can provide an interactive connection for example for subscribed members transactions using subscribed members digital devices including a smart phone 510 with the rewards program digital device application 400 installed; a tablet 520 with the rewards program digital device application 400 installed; a laptop computer 530 with the rewards program digital device application 400 installed; and a computer 540 with the rewards program digital device application 400 installed of one embodiment.

### Rewards Program Member Memberships:

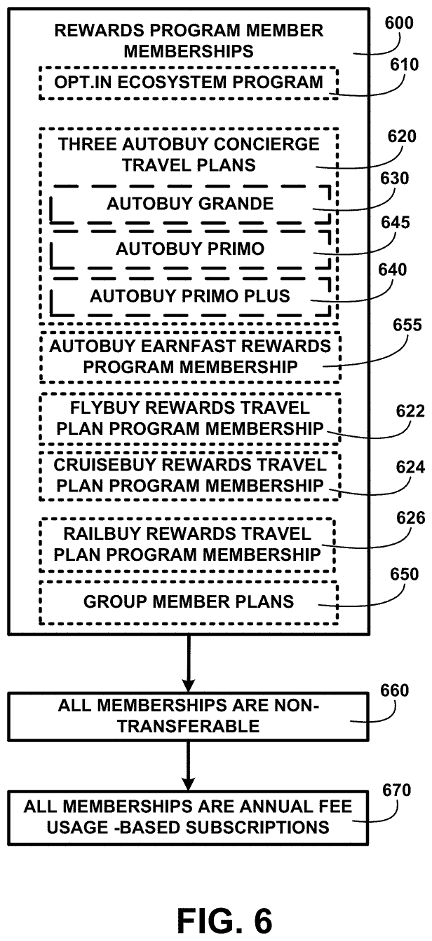

FIG. 6 shows a block diagram of an overview of rewards program member memberships of one embodiment. FIG. 6 shows rewards program member memberships 600 that a subscribed member can select including an Opt.In Ecosystem program 610; three concierge travel plans 620 including Grande 630, PRIMO 645, and PRIMO PLUS 640; AutoBuy Earnfast Rewards Program membership 655; FlyBuy Rewards Travel Plan Program membership 622; CruiseBuy Rewards Travel Plan Program membership 624, RailBuy Rewards Travel Plan Program membership 626 and group member plans 650 and other member plans. All memberships are non-transferable 660. All memberships are annual fee usage-based subscriptions 670 of one embodiment.

### Self-Reported Data of Activities of Daily Living:

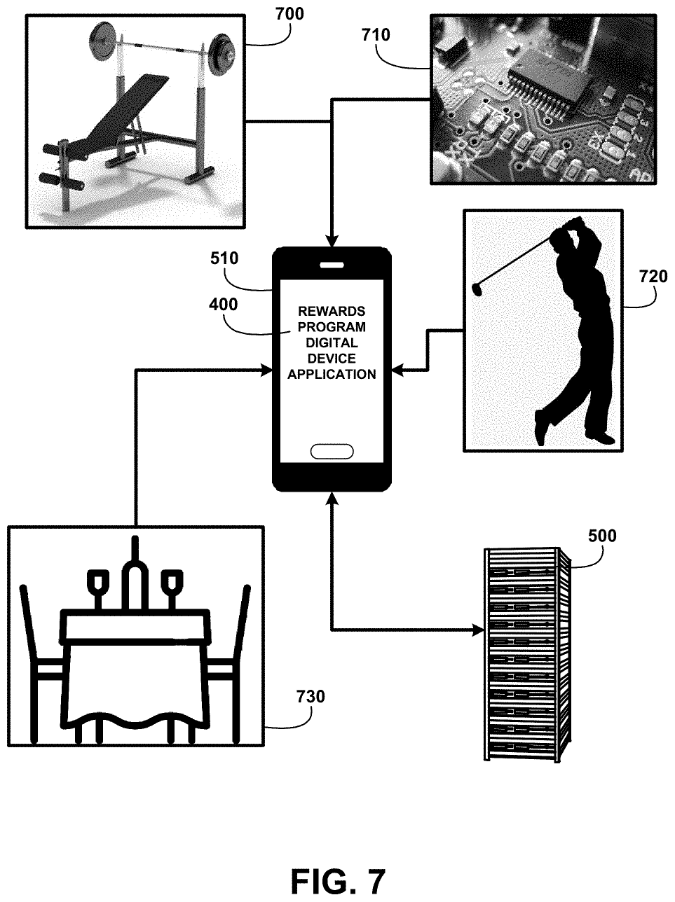

FIG. 7 shows for illustrative purposes only an example of self-reported data OF activities of daily living of one embodiment. FIG. 7 shows the subscribed member smart phone 510 with the rewards program digital device application 400 installed. The rewards program digital device application 400 is uploading to the at least one rewards program server 500 the subscribed members' activities including for example going to a fitness club 700; participating in an electronics hobby 710; playing golf 720 and enjoying fine dining 730 at an upscale restaurant. The rewards program digital device application 400 is collecting authorized Opt-In self-reported data including the subscribed members' activities of daily living, hobbies, likes and dislikes. The rewards program digital device application 400 can also be used for collecting the members tax returns and perform GPS tracking of where the member is going on a daily basis of one embodiment.

### Health Risk Assessment:

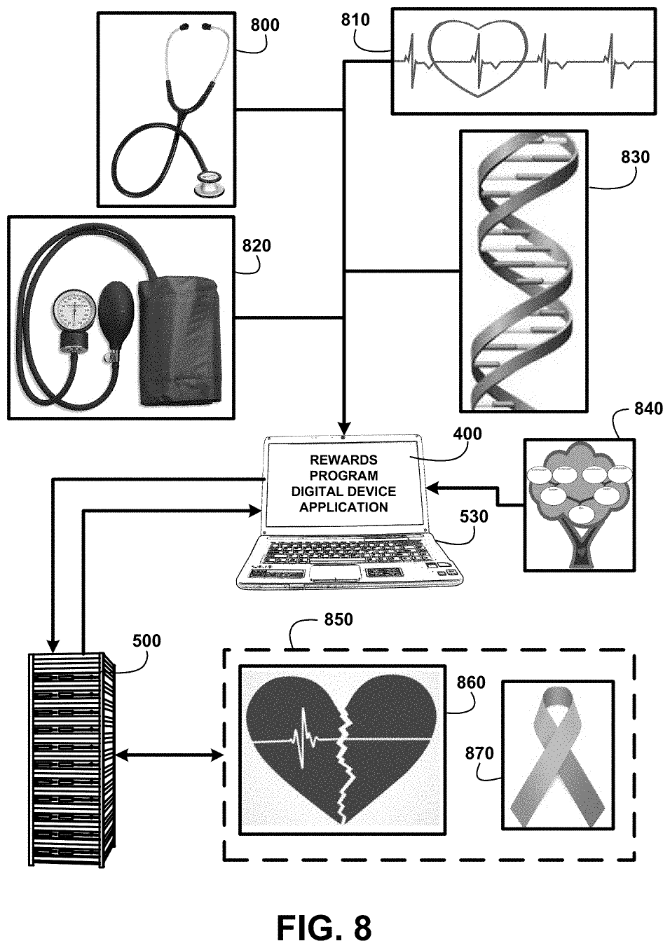

FIG. 8 shows for illustrative purposes only an example of health risk assessment of one embodiment. FIG. 8 shows the subscribed members' laptop computer 530 with the rewards program digital device application 400 installed communicating with the at least one rewards program server 500 via the at least one internet connectable device 582. The subscribed member can be self-reporting their health condition 800; cardio lab tests 810; blood pressure 820; DNA results 830; and family tree and history 840; background for example any military duty, job history which for example may indicate potential exposure to hazardous materials, other lab tests and hereditary DNA tests, ancestry DNA tests, and blood tests.

The at least one rewards program server 500 using the at least one digital processor 560 can perform a subscribed members' health risk assessment 850 using the self-reported health condition 800 and other health related data to perform a heart condition analysis 860 based on the DNA results 830; family tree and history 840; blood pressure 820 and cardio lab tests 810, background for example any military duty, job history which for example may indicate potential exposure to hazardous materials, other lab tests and hereditary DNA tests, ancestry DNA tests, and blood tests. The at least one rewards program server 500 using the at least one printer 555 can print the heart condition analysis 860 for example in a .pdf format and email the results to the subscribed member.

The at least one rewards program server 500 using the at least one digital processor 560 can perform a subscribed members' health risk assessment 850 using the self-reported health condition 800 and other health related data to perform a breast cancer analysis 870 based on the DNA results 830; and family tree and history 840; background for example any military duty, job history which for example may indicate potential exposure to hazardous materials, other lab tests and hereditary DNA tests, ancestry DNA tests, and blood tests. The at least one rewards program server 500 using the at least one printer 555 can print the breast cancer analysis 870 for example in a hard copy format and mail the results to the subscribed member for example for the member to share with their physician. The health risk assessment personalized for the subscribed member may provide them with early health risks alerts they were unaware of and could lead to early detection and treatment of a potentially serious health condition of one embodiment.

### Tracking Your Social Media:

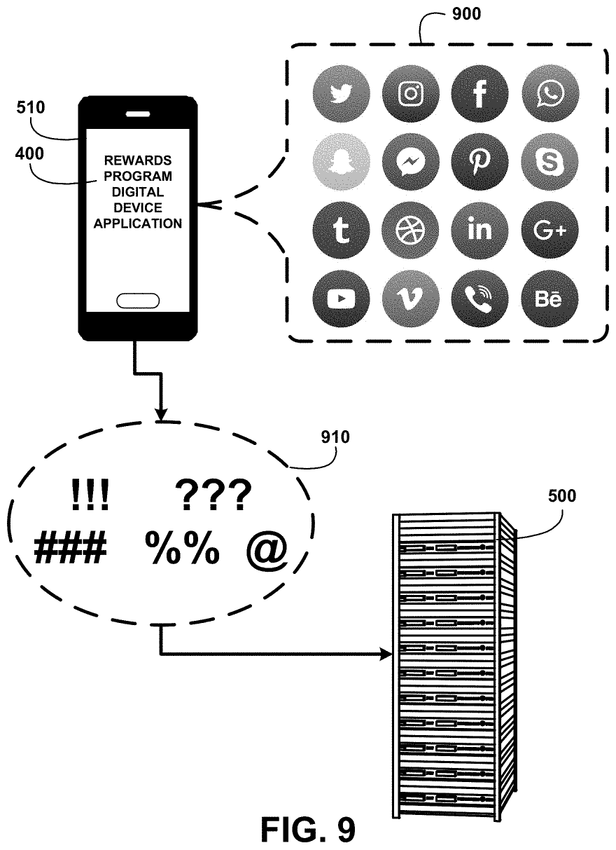

FIG. 9 shows for illustrative purposes only an example of tracking your social media of one embodiment. FIG. 9 shows the subscribed members' smart phone 510 with the rewards program digital device application 400 installed providing a connection with the at least one rewards program server 500 for collecting authorized social media dialogues 900 and social media comments 910 of the member and their family, friends and associates. Tracking a members' social media provide an analysis opportunity to determine what areas a member and their friends talk about and debate about. The social media analysis can pinpoint additional likes and dislikes of a subscribed member to direct for example rewards points redemption opportunities that fit into their personalized likes and dislikes and potential bucket list interest.

The social media analysis can be performed by the at least one rewards program server 500; at least one digital database 550 using recorded member personalized data; at least one digital processor 560 analytics of the at least one OCR device 570 parsed content of the social media subjects talked about and debated about and recorded in the database using the voice recognition and transcription device 584. The at least one language translation apparatus 572 can be used to confirm specific meaning of a term or phrase in a language to arrive at any difference associated with a local nuance ascribed to that particular term or phrase of one embodiment.

### Concierge Travel Plans:

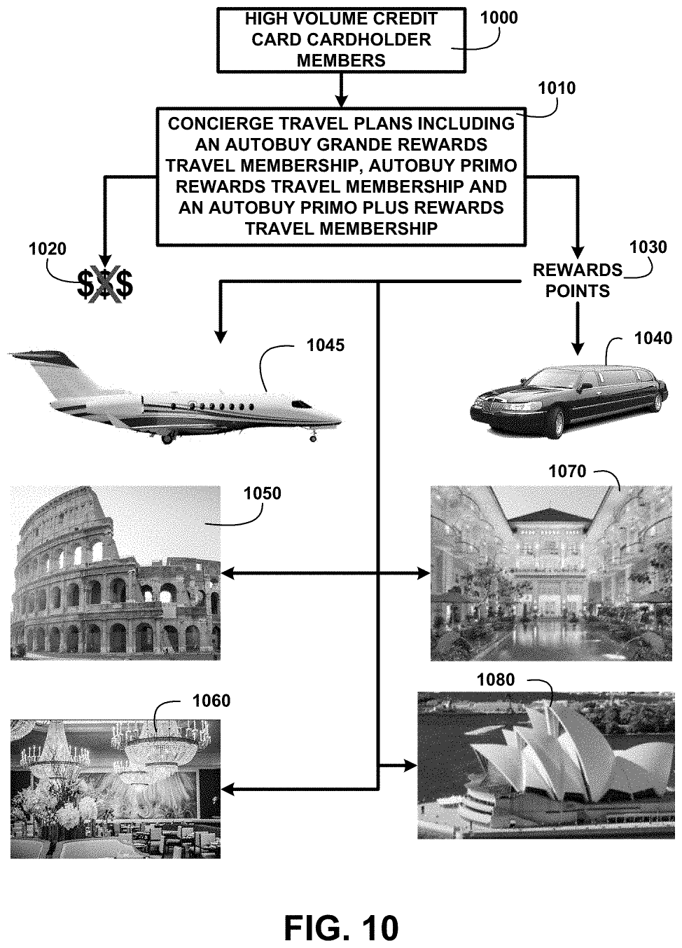

FIG. 10 shows for illustrative purposes only an example of concierge travel plans of one embodiment. FIG. 10 shows high volume credit card cardholder members 1000 may accumulate adequate AutoBuy rewards points for redemption using one of the AutoBuy rewards concierge travel plans including an AutoBuy Grande Rewards Travel Membership; AutoBuy Primo Rewards Travel Membership; and an AutoBuy Primo Plus Rewards Travel Membership 1010. The rewards travel memberships are subscription plans wherein a subscribed member can enjoy reduced to no cash expense 1020 and use rewards points 1030 to purchase contracted company travel opportunities. For example a rewards travel member can use points accumulated to pay the expenses of Shared Private Jet transportation travel 1045 including international travel 1050. Rewards travel benefits can also include ground transportation 1040 including limousine service; hotel accommodations 1070; fine dining 1060; and entertainment 1080 at world class events and facilities of one embodiment.

These three special memberships are only available when redeeming AutoBuy Reward Points. If the Shared Private Jet travel exceeds a members' AutoBuy Reward Points they are able to pay the balance with any contracted credit card. These three Concierge Memberships are designed to be used by families who travel on a regular basis for either business or pleasure. These are also designed for small, medium and large businesses, corporations and governments who don't have a personal or corporate agency or travel department. These memberships are designed to provide access to a Rewards program professional concierge. These professionals will assist you in securing personal or business services, plan a family trip, and secure plane flights with one of the Rewards programs Shared Private Jet partner firms.

AutoBuy GRANDE Rewards Travel members can access a professional concierge 16 hours a day 365 days a year. The annual subscription fee is payable by a single or double party membership and a Group Member Plan for up to six members. A company is allowed to purchase as many Group Member Plans as they need.

AutoBuy PRIMO Rewards Travel Members have access to the Rewards program professional concierge 24 hours a day and 365 days a year. AutoBuy PRIMO Rewards Travel Members can enjoy concierge arranged domestic and international flights with one of the Rewards programs Shared Private Jet partner firms, a yacht at their destination, a villa rental, seaside village accommodations and other expanded travel opportunities.

AutoBuy PRIMO PLUS Rewards Travel Memberships contain additional benefits including services with a Rewards authorized OEM Automotive Dealership partner to assist in the purchase and bidding process at any auto auction worldwide. Auto and vehicle equipment auctions are held worldwide and this service will allow a PRIMO PLUS member to bid without traveling to each and every auction venue. This service can also include the OEM Automotive Dealership partner assisting in the vetting of the auto or equipment, securing average pricing for like vehicles, transportation of purchased vehicles through transport industry partners, using a Pooling of Points (POP) program and any number of other services.

FlyBuy Rewards Travel Plan Program membership 622 of FIG. 6 can earn a member points for air travel on participating airlines in addition to any frequency flyer points they receive from the air line. Additional points can be earned by a member for credit card purchases from participating vendors for example at an airport restaurant, buying luggage from a participating retailer including international airport participating vendors. The member can increase travel opportunities with the added benefit of redemption of FlyBuy Rewards points of one embodiment.

CruiseBuy Rewards Travel Plan Program membership 624 of FIG. 6 can earn a member points for shipboard travel on participating cruise lines. Additional points can be earned by a member for credit card purchases from participating vendors for example while in port including international ports from for example participating restaurants, buying souvenirs from a participating retailer and purchasing cruise clothing from a participating clothing store. The member can increase travel opportunities with the added benefit of redemption of CruiseBuy Rewards points of one embodiment.

RailBuy Rewards Travel Plan Program membership 626 of FIG. 6 can earn a member points for rail line travel on participating Rail lines. Additional points can be earned by a member for credit card purchases from participating vendors for example café cars on a train while riding the rails from city to city. More points can be earned by a member for credit card purchases on stop overs from for example participating restaurants, buying souvenirs from a participating retailer and participating hotels on overnight enroute stopovers. The member can increase travel opportunities with the added benefit of redemption of RailBuy Rewards points of one embodiment.

A Rewards member first annual Opt.In membership fee will be paid upon joining with the credit card that will connect with the AutoBuy Rewards Membership program. After the first annual fee the membership annual fee can be automatically debited to the members' Personal Rewards Points Redemption Account with the member receiving an email showing the payment debited from the members account. Rewards concierge travel contact can be with a professional concierge fluent in multiple languages.

### Tracking Your Visits:

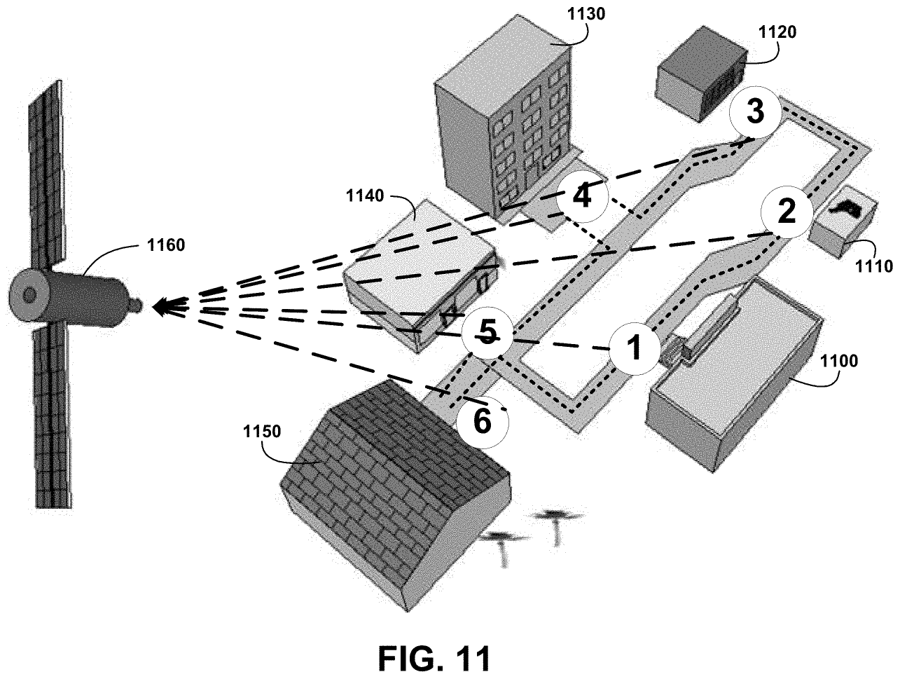

FIG. 11 shows for illustrative purposes only an example of tracking your visits of one embodiment. FIG. 11 shows the rewards program digital device application 400 being used for tracking an Opt-In subscribed member visits to destinations to rank their favorites and most frequent destinations. The rewards program digital device application 400 can use the GPS location of a members' smart phone 510 and satellite GPS locator 1160 systems to record daily activities.

A member's daily activities may include paying for various wants and necessities for example a medication prescription. A participating vendor may include a contracted participating pharmacy where a member purchase using the member Opt-In credit card will earn the member additional points. The accumulated participating pharmacy points can be for example redeemed for future purchases from the participating pharmacy.

A members' daily activities can include for example a trip to a #1 library 1100 to research a favorite subject, author or genre of literature; #2 pharmacy 1110 to pick up a prescription that may be available through a contracted pharmacy company where the member can use rewards points to pay for the prescription; and #3 sporting goods store 1120 to pick up tennis equipment that may be available through a contracted sporting goods company where the member can redeem rewards points for the purchase. The Opt-In subscribed member may visit a #4 doctors office 1130 to consult on a health risk assessment analysis alert; then proceed to a #5 grocery store 1140 to pick up groceries for example that may include food and beverage items that may be available by the member redeeming rewards points at participating food outlets; and finally returning to #6 home 1150.

A doctor may be contracted with a health care company that is contracted as a participating vendor. The cost of health is a major concern of any potential member. Health care companies may choose to contract with the rewards program method and devices programs to assist their patients in a method of reducing out of pocket health expenses. Health insurers generally do not cover all medical costs. A patient that pays any co-pays or non-covered medical expenses with the Opt-In credit card can accumulate points for future medical expenses. Contracting with health care providers adds a direct benefit to the health risk assessment of the Opt-In Rewards program. Not only will the Opt-In Rewards program alert a member of any potential health risk but additionally provide a vehicle to assist in paying for any consultations and treatments should the health risk materialize.

The member gains the benefit of point redemption versus out of pocket cash expense and the health care provider gains positive public relations with the particular patient and others showing they are also concerned with the cost of health care and are actually doing something to provide some relief to at least Opt-In member credit cardholders of one embodiment.

### AutoBuy Earnfast Rewards Program Benefits:

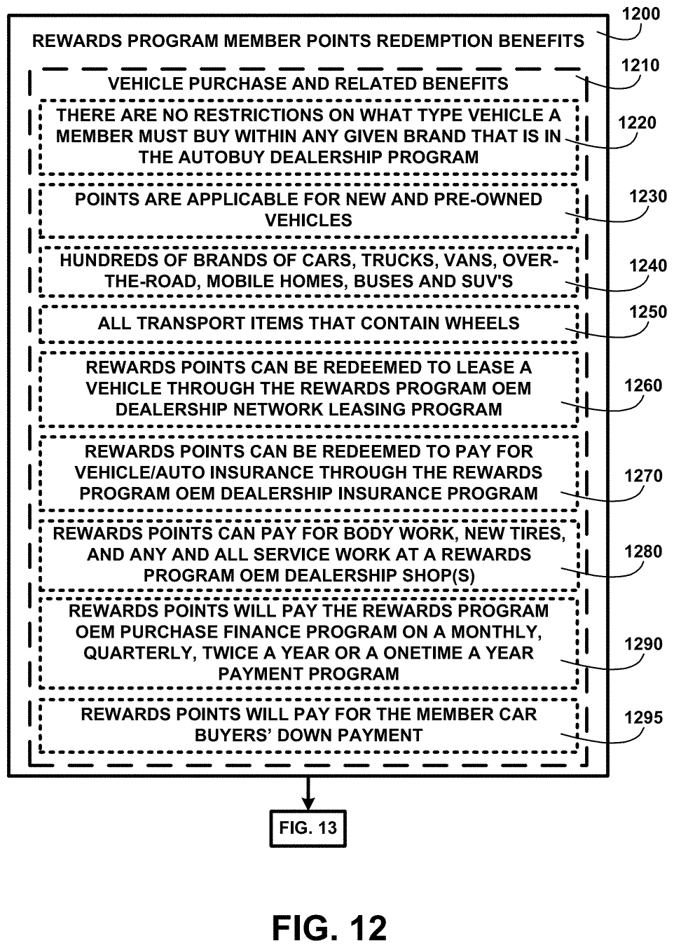

FIG. 12 shows a block diagram of an overview of AutoBuy Earnfast Rewards Program benefits of one embodiment. FIG. 12 shows AutoBuy Earnfast Rewards Program benefits which the member can subscribe to as one rewards program member points redemption benefits 1200 programs. The AutoBuy Earnfast Rewards Program benefits includes a vehicle purchase and related benefits 1210. There are no restrictions on what type vehicle a member must buy within any given brand that is in the AutoBuy dealership program 1220. Points are applicable for new and pre-owned vehicles 1230 including hundreds of brands of cars, trucks, vans, over-the-road, mobile homes, buses and SUV's 1240 and all transport items that contain wheels 1250.

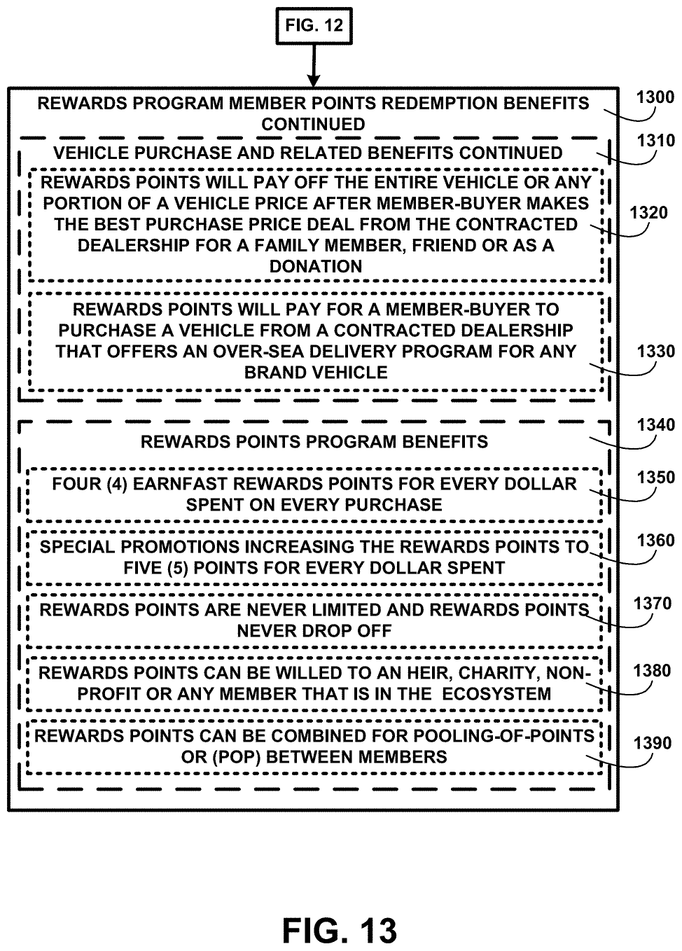

Rewards points can be redeemed to lease a vehicle through the rewards program OEM dealership network leasing program 1260. Rewards points can be redeemed to pay for vehicle/auto insurance through the rewards program OEM dealership insurance program 1270. Rewards points can pay for body work, new tires, and any and all service work at a rewards program OEM dealership shop(s) 1280. Rewards points will pay the rewards program OEM purchase finance program on a monthly, quarterly, twice a year or a onetime a year payment program 1290. Rewards points will pay for the member car buyers' down payment 1295 of one embodiment. The descriptions continue on FIG. 13.

### Vehicle Purchase and Related Benefits:

FIG. 13 shows a block diagram of an overview of vehicle purchase and related benefits of one embodiment. FIG. 13 shows continuing from FIG. 12 the rewards program member Rewards Points redemption benefits continued 1300 and vehicle purchase and related benefits continued 1310. Rewards points will pay off the entire vehicle or any portion of a vehicle price after member-buyer makes the best purchase price deal from the contracted dealership for a family member, friend or as a donation 1320. Rewards points will pay for a member-buyer to purchase a vehicle from a contracted dealership that offers an over-sea delivery program for any brand vehicle 1330. Rewards points program benefits 1340 include four (4) earned rewards points for every dollar spent on every purchase 1350. Special promotions increasing the rewards points to five (5) points for every dollar spent 1360. Rewards points are never limited and rewards points never drop off 1370. Rewards points can be willed to an heir, charity, non-profit or any member that is in the Ecosystem 1380. Rewards points can be combined for Pooling-Of-Points or (POP) between members 1390 of one embodiment.

### Community Banking System Participant Benefits:

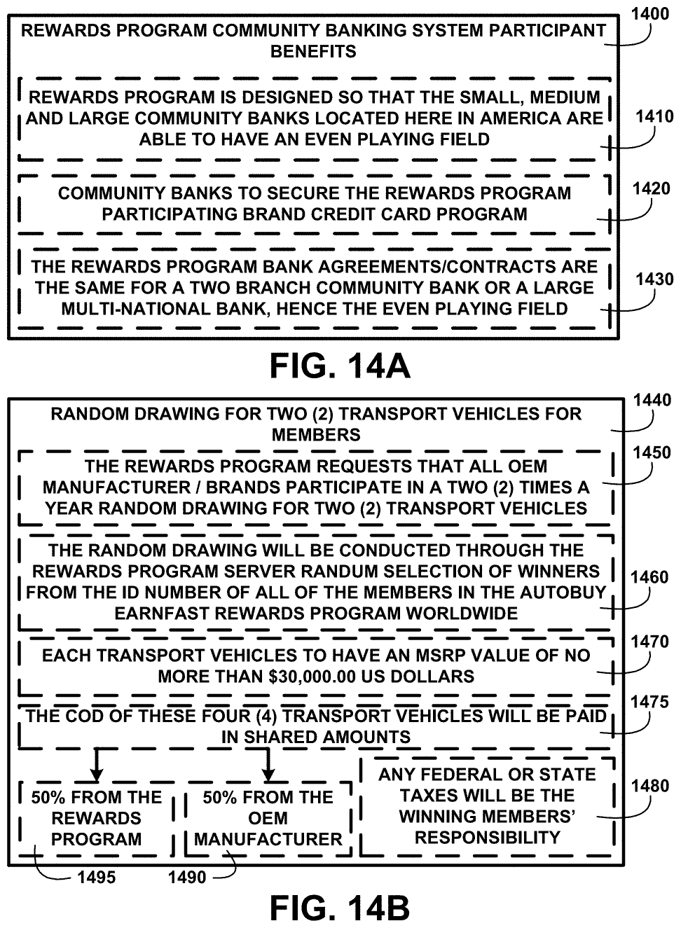

FIG. 14A shows a block diagram of an overview of community banking system participant benefits of one embodiment. FIG. 14A shows a rewards program community banking system participant benefits 1400. The rewards program is designed so that the small, medium and large community banks located here in America is able to have an Even Playing Field 1410 for community banks to secure the rewards program participating brand credit card program 1420. The rewards program bank agreements/contracts are the same for a two branch community bank or a large multi-national bank, hence the Even Playing Field 1430 of one embodiment.

### Random Drawing for Members:

FIG. 14B shows a block diagram of an overview of random drawing for members of one embodiment. FIG. 14B shows a random drawing for two (2) transport vehicles for members 1440. The rewards program requests that all OEM manufacturer/brands participate in a two (2) times a year random drawing for two (2) transport vehicles 1450. The random drawing will be conducted through the rewards program server random selection of winners from the ID number of all of the members in the AutoBuy Earnfast Rewards Program worldwide 1460. Each transport vehicles to have an MSRP value of no more than $30,000.00 US dollars 1470. The COD of these four (4) transport vehicles will be paid in shared amounts 1475 of 50% from the rewards program 1495 and 50% from the OEM manufacturer 1490. Any federal or state taxes will be the winning members' responsibility 1480 of one embodiment.

### Opt.In Ecosystem Program:

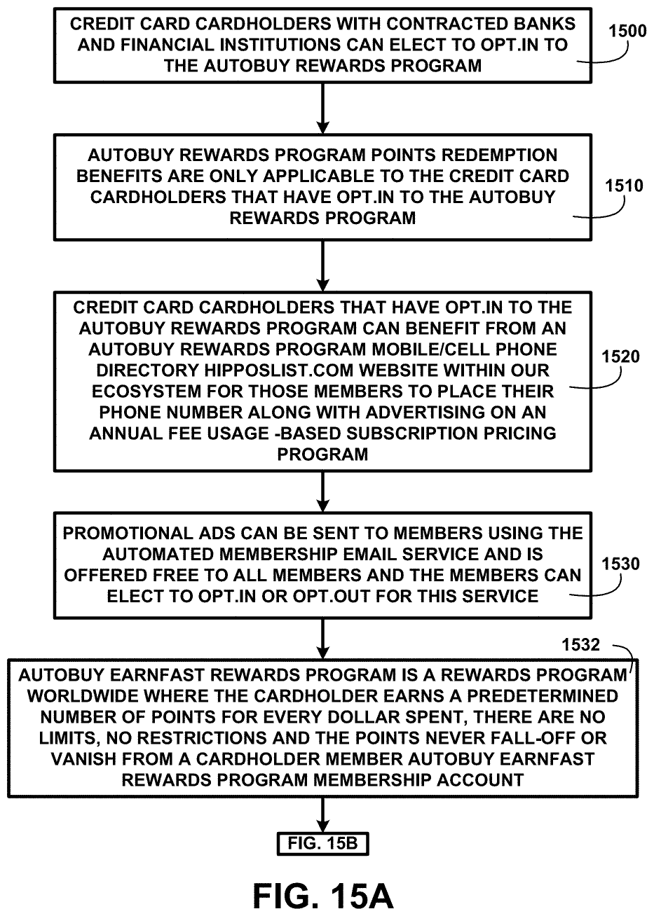

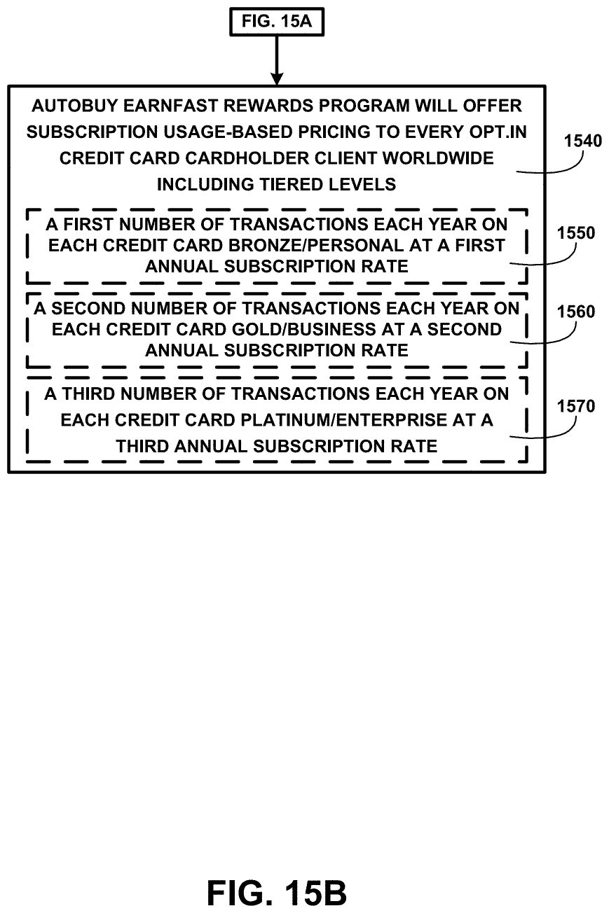

FIG. 15A shows a block diagram of an overview of Opt.In Ecosystem program of one embodiment. FIG. 15A shows credit card cardholders with contracted banks and financial institutions can elect to Opt.In to the AutoBuy Rewards program 1500. AutoBuy rewards program points redemption benefits are only applicable to the credit card cardholders that have Opt.In to the AutoBuy Rewards program 1510. Credit card cardholders that have Opt.In to the AutoBuy Rewards program can benefit from an AutoBuy Rewards program mobile/cell phone directory Hipposlist.com website within our Ecosystem for those members to place their phone number along with advertising on an annual fee usage-based subscription pricing program 1520. Promotional ads can be sent to members using the automated membership email service and is offered free to all members and the members can elect to Opt.In or Opt.Out for this service 1530. AutoBuy Earnfast Rewards Program is a rewards program worldwide where the cardholder earns a predetermined number of points for every dollar spent, there are no limits, no restrictions and the points never fall-off or vanish from a cardholder member AutoBuy Earnfast Rewards Program membership account 1532 of one embodiment. Additional descriptions continue on FIG. 15B.

### AutoBuy Earnfast Rewards Program Worldwide:

FIG. 15B shows a block diagram of an overview of AutoBuy Earnfast Rewards Program worldwide of one embodiment. FIG. 15B shows continuing from FIG. 15A that AutoBuy Earnfast Rewards Program will offer subscription usage-based pricing to every Opt.In credit card cardholder client worldwide including tiered levels 1540. AutoBuy Earnfast Rewards Program subscription tiered levels include a first number of transactions each year on each credit card bronze/personal at a first annual subscription rate 1550; a second number of transactions each year on each credit card gold/business at a second annual subscription rate 1560; and a third number of transactions each year on each credit card platinum/enterprise at a third annual subscription rate 1570 of one embodiment.

### AutoBuy Rewards Program Revenue Streams:

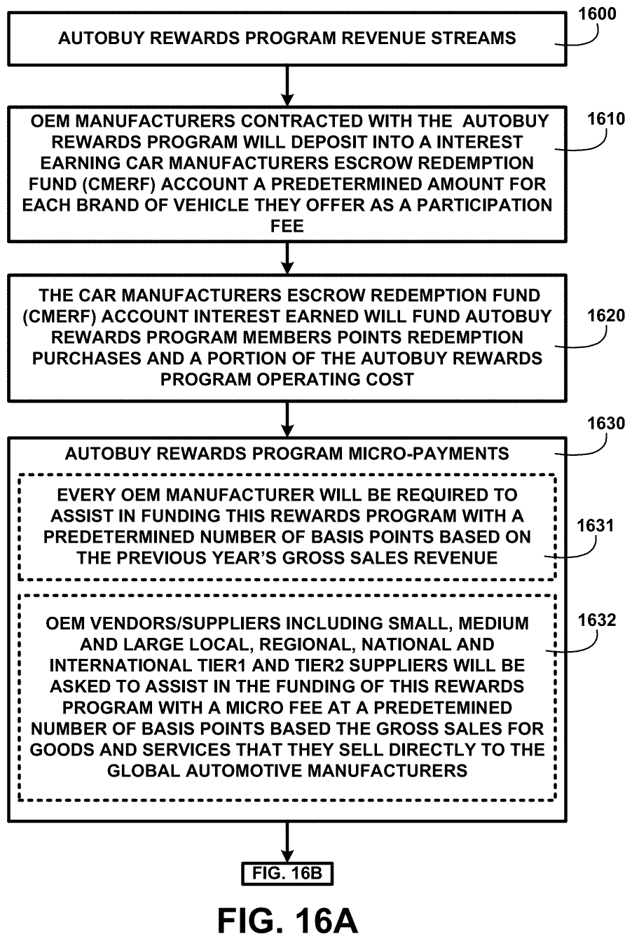

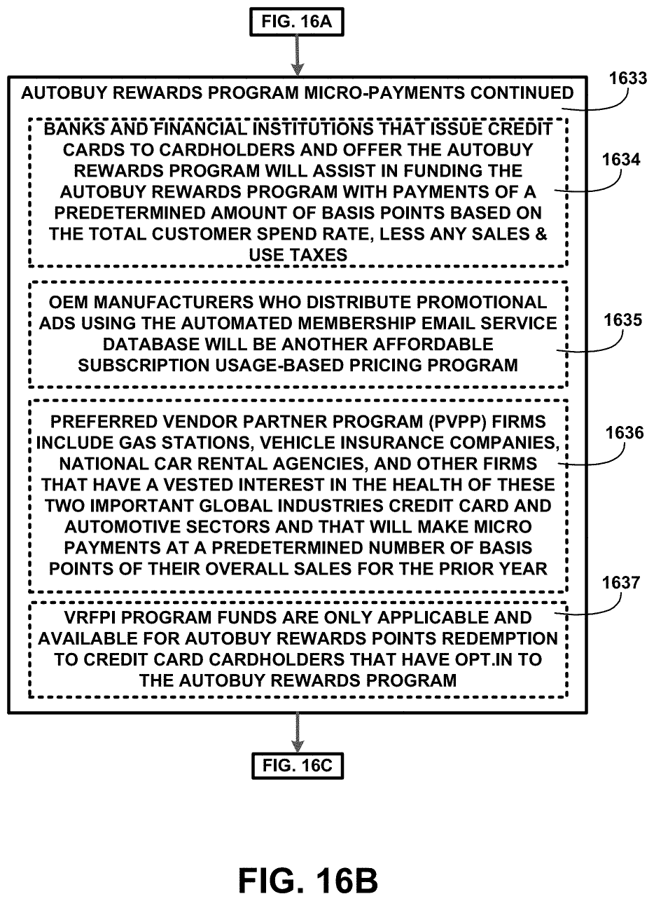

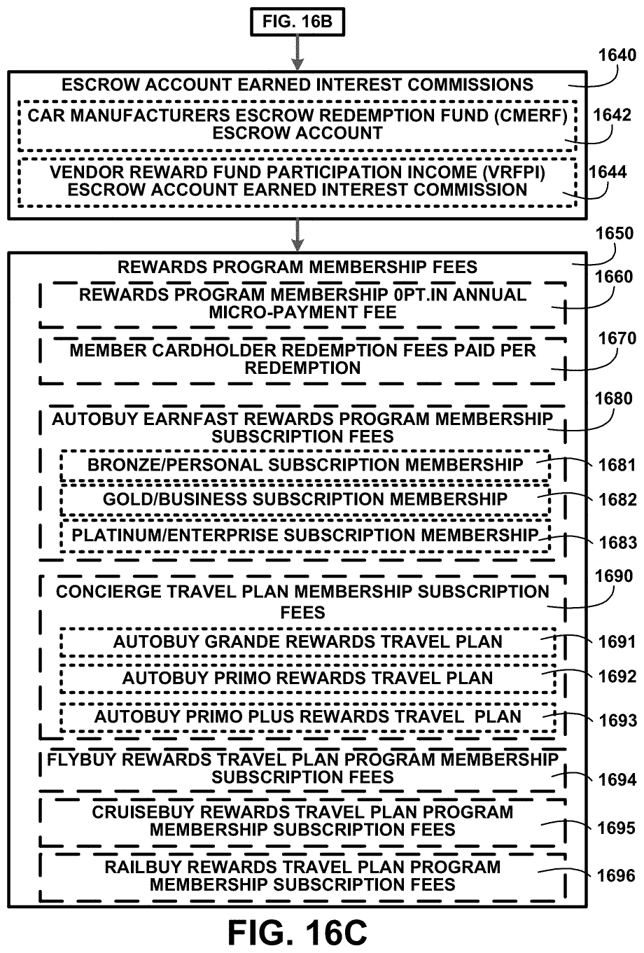

FIG. 16A shows a block diagram of an overview of AutoBuy Rewards program revenue streams of one embodiment. FIG. 16A shows some embodiments of AutoBuy Rewards program revenue streams 1600. AutoBuy rewards program revenue streams 1600 can include a revenue stream where OEM manufacturers contracted with the AutoBuy Rewards program will deposit into an interest earning Car Manufacturers Escrow Redemption Fund (CMERF) escrow account a predetermined amount for each brand of vehicle they offer as a participation fee 1610.

The at least one rewards program server 500 of FIG. 5; at least one digital database 550 of FIG. 5; at least one digital processor 560 of FIG. 5 can be configured for recording and monitoring funding from contracted companies including interest earning from the Car Manufacturers Escrow Redemption Fund (CMERF) escrow account. The Car Manufacturers Escrow Redemption Fund (CMERF) escrow account interest earned will fund AutoBuy Rewards program members' points redemption purchases and a portion of the AutoBuy Rewards program operating cost 1620 of one embodiment.

AutoBuy rewards program micro-payments 1630 are another embodiment of the revenue streams. Every OEM manufacturer will be required to assist in funding this rewards program with a predetermined number of basis points based on the previous year's gross sales revenue 1631. OEM vendors/suppliers including small, medium and large local, regional, national and international tier1 and tier2 suppliers will be asked to assist in the funding of this rewards program with a micro fee at a predetermined number of basis points based the gross sales for goods and services that they sell directly to the global automotive manufacturers 1632. Additional descriptions of micro-payments are on FIG. 16B.

### AutoBuy Rewards Program Micro-Payments Continued:

FIG. 16B shows a block diagram of an overview of AutoBuy Rewards program micro-payments continued of one embodiment. FIG. 16B shows a continuation from FIG. 16A with AutoBuy Rewards program micro-payments continued 1633. Banks and financial institutions that issue credit cards to cardholders and offer the AutoBuy Rewards program will assist in funding the AutoBuy Rewards program with payments of a predetermined amount of basis points based on the total customer spend rate, less any sales & use taxes 1634. OEM manufacturers who distribute promotional ads using the automated membership email service database will be another affordable subscription usage-based pricing program 1635.

Preferred Vendor Partner Program (PVPP) firms include gas stations, vehicle insurance companies, national car rental agencies, and other firms that have a vested interest in the health of these two important global industries credit card and automotive sectors and that will make micro payments at a predetermined number of basis points of their overall sales for the prior year 1636. VRFPI program funds are only applicable and available for AutoBuy Rewards points redemption to credit card cardholders that have Opt.In to the AutoBuy Rewards program 1637 of one embodiment. Descriptions of more revenue streams continue on FIG. 16C.

### Escrow Account Earned Interest Commissions:

FIG. 16C shows a block diagram of an overview of escrow account earned interest commissions of one embodiment. FIG. 16C shows a continuation from FIG. 16B of revenue streams with escrow account earned interest commissions 1640. Escrow account earned interest commissions 1640 include a predetermined percentage of the interest earned from the Car Manufacturers Escrow Redemption Fund (CMERF) escrow account 1642 and the Vendor Reward Fund Participation Income (VRFPI) escrow account earned interest commission 1644. The at least one rewards program server 500 of FIG. 5; at least one digital database 550 of FIG. 5; at least one digital processor 560 of FIG. 5 can be configured for recording and monitoring funding from contracted companies including interest earning from the Vendor Reward Fund Participation Income (VRFPI) escrow account of one embodiment.

Rewards program membership fees 1650 are another source of revenue streams. Rewards program membership fees 1650 include a Rewards program membership Opt.in annual micro-payment fee 1660. Another revenue source is Member cardholder redemption fees paid per redemption 1670. AutoBuy Earnfast Rewards Program membership subscription fees 1680 including the Bronze/personal subscription membership 1681; Gold/business subscription membership 1682; and Platinum/enterprise subscription membership 1683. Another revenue stream is generated with Concierge travel plan membership subscription fees 1690 including an AutoBuy GRANDE rewards travel plan 1691; AutoBuy PRIMO rewards travel plan 1692; AutoBuy PRIMO PLUS rewards travel plan 1693; FlyBuy Rewards travel plan program membership subscription fees 1694; CruiseBuy Rewards travel plan program membership subscription fees 1695, and RailBuy Rewards travel plan program membership subscription fees 1696 of one embodiment.

The foregoing has described the principles, embodiments and modes of operation of the embodiments. However, the embodiments should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

1. A method, comprising:
creating a digital network for processing and analyzing credit card holder member data for establishing a worldwide rewards program with interactive services and functions to members;
wherein the digital network consists of coupled processing devices consisting of a group of at least one rewards program server, at least one digital database, at least one digital processor, at least one OCR device, at least one WI-FI connectable device, at least one printer at least one video device, at least one language translation apparatus, at least one data mining device, at least one internet connectable device, voice recognition and transcription device, at least one email server, and an automated membership email service database;
contracting with companies on a participation basis for funding a credit card holder member rewards;
purchasing products and services by a member using accumulated rewards program points and purchasing from the contracted companies to reduce member out of pocket purchasing expenses;
using the digital network configured to include at least one rewards program server and at least one digital processor coupled to at least one internet connectable device for connecting to a member's social media applications for tracking a member's social media communications to perform an analysis using at least one digital processor for analyzing additional likes and dislikes of a subscribed member to direct to the member rewards points redemption opportunities;
using the at least one language translation apparatus to confirm a specific meaning of a social media communications term or phrase to arrive at any difference associated with a local nuance ascribed to that particular term or phrase for use in analyzing additional likes and dislikes of a subscribed member;
offering purchases of contracted companies products including vehicles using credit card holder member points for redemption purchases; and
performing a health risk assessment analysis including but not limited to performing a breast cancer analysis based on DNA results hereditary DNA tests, ancestry DNA tests and performing a heart condition analysis based on DNA results hereditary DNA tests, ancestry DNA tests for detecting early health risks a member may be unaware of and could lead to early detection and treatment of a potentially serious health condition.

2. The method of claim 1, further comprising using at least one digital processor for determining a predetermined portion of interest earned In contracted companies participation funds escrow accounts for funding the credit card holder member points for redemption purchases.

3. The method of claim 1, further comprising using at least one digital processor for determining a predetermined portion of interest earned in a Car Manufacturers Escrow Redemption Fund escrow account to fund credit card holder member points for redemption purchases of vehicles from contracted OEM automotive manufacturer participants.

4. The method of claim 1, further comprising using at least one digital processor for determining what portion of interest earned in a Vendor Reward Fund Participation Income escrow account is used to fund credit card holder member points for redemption purchases of vehicles, vehicle/auto insurance through a rewards program OEM dealership insurance program, body work, new tires, and any and all service work.

5. The method of claim 1, further comprising using the member's social media tracking analysis additional likes and dislikes of a subscribed member for offering credit card cardholder members subscriptions to a concierge travel plan including tiered travel plan programs for redeeming member points for expenses of Shared Private Jet transportation travel including international travel, ground transportation including limousine service; hotel accommodations, fine dining; and entertainment at world class events and facilities.

6. The method of claim 1, further comprising using the digital network processing and analyzing of credit card holder member data including tracking member's social media communications for determining subscribed members additional likes and dislikes to direct to the member rewards points redemption opportunities from contracted companies in a Preferred Vendor Partner Program and including credit card companies, banks issuing credit cards, and other consumer product/service providers including transportation, retail, service, travel, hospitality, food service, gas stations, vehicle insurance companies, national car rental agencies, and other firms that have a vested interest in the health of these two important global industries credit card and automotive sectors.

7. The method of claim 1, further comprising using a digital network configured to include at least one rewards program server and server computer for controlling coupled processing digital devices including the at least one email server and the automated membership email service database for member email delivery of contracted company promotional ads.

8. The method of claim 1, further comprising tracking a subscribed member's social media using the at least one rewards program server, using at least one digital database recorded subscribed member personalized data; and at least one digital processor analytics of at least one OCR device parsed content social media subjects talked about and debated about and recorded in the at least one digital database using voice recognition and transcription devices to analyze additional likes and dislikes of a subscribed member to direct rewards points redemption opportunities that fit into their personalized likes and dislikes and potential bucket list interest.

9. The method of claim 1, further comprising using at least one digital database configured to record, retrieve and analyze the personalized member data to determine a subscribed members' health risk assessment including using at least one rewards program server using at least one digital processor to perform a members' health risk assessment including performing a breast cancer analysis based on DNA results; and family tree and history; background including any military duty, job history indicating potential exposure to hazardous materials, other lab tests and hereditary DNA tests, ancestry DNA tests, and blood tests and performing a heart condition analysis based on the DNA results; family tree and history; blood pressure and cardio lab tests, background for example any military duty, job history which for example may indicate potential exposure to hazardous materials, other lab tests and hereditary DNA tests, ancestry DNA tests, and blood tests for detecting early health risks a member may be unaware of and could lead to early detection and treatment of a potentially serious health condition.

10. The method of claim 1, further comprising using a GPS location device of a members' smart phone with the rewards program digital device application installed and satellite GPS locator systems for GPS tracking of where a member goes on a daily basis.

11-20. (canceled)
